(19) United States
(12) Patent Application Publication
(10) Pub. No.: US 2025/0066445 A1
(43) Pub. Date: Feb. 27, 2025

(54) OBESITY TREATMENT AND PREVENTION METHODS

(71) Applicant: VAULT BIOSCIENCES, INC., San Diego, CA (US)

(72) Inventor: Joe A. YOUNG, Carlsbad, CA (US)

(21) Appl. No.: 18/942,271

(22) Filed: Nov. 8, 2024

Related U.S. Application Data
(63) Continuation of application No. 18/199,856, filed on May 19, 2023.
(60) Provisional application No. 63/343,823, filed on May 19, 2022.

(51) Int. Cl. C07K 14/605; A61P 3/04

(52) U.S. Cl. CPC C07K 14/605; A61P 3/04

(57) ABSTRACT

Provided herein are methods for treating or preventing obesity and obesity related diseases and disorders. In some embodiments, the methods comprise providing a glucagon-like peptide-1 (GLP-1) receptor agonist with a pharmaceutically or nutritionally acceptable carrier to the patient at a maintenance dose lower than a lowest therapeutic dose (LTD) to be administered during a first period, wherein during a second period that overlaps with the first period, the subject is on an insulin modulating nutritional therapy protocol.

### CROSS-REFERENCE TO RELATED APPLICATIONS

This application is a continuation of U.S. application Ser. No. 18/199,856, filed on May 19, 2023, which claims priority to U.S. Provisional Application Ser. No. 63/343,823, filed on May 19, 2022, which is incorporated by reference in its entirety.

### BACKGROUND

Obesity, including abdominal obesity, is associated with an increased risk of many serious diseases and disorders and/or exacerbating symptoms or risk of progression of many serious diseases and disorders, including type 2 diabetes, coronary heart disease, hypertension, stroke, osteoarthritis, sleep apnea, and polycystic ovarian syndrome.

### SUMMARY OF THE DISCLOSURE

In an aspect, a method for treating or preventing obesity in a subject is provided, comprising providing a glucagon-like peptide-1 (GLP-1) receptor agonist with a pharmaceutically or nutritionally acceptable carrier to the patient at a maintenance dose lower than a lowest therapeutic dose (LTD) to be administered during a first period, wherein during a second period that overlaps with the first period, the subject is on an insulin modulating nutritional therapy protocol.

In some embodiments, the maintenance dose is between 10%-95% of the LTD for the GLP-1 receptor agonist. In some embodiments, the maintenance dose is between 65%-95% of the LTD for the GLP-1 receptor agonist. In some embodiments, the maintenance dose is between 70%-90% of the LTD for the GLP-1 receptor agonist. In some embodiments, the maintenance dose is between 70%-80% of the LTD for the GLP-1 receptor agonist. In some embodiments, the maintenance dose is between 35%-65% of the LTD for the GLP-1 receptor agonist. In some embodiments, the maintenance dose is between 40%-60% of the LTD for the GLP-1 receptor agonist. In some embodiments, the maintenance dose is between 45%-55% of the LTD for the GLP-1 receptor agonist. In some embodiments, the maintenance dose is between 10%-35% of the LTD for the GLP-1 receptor agonist. In some embodiments, the maintenance dose is between 15%-30% of the LTD for the GLP-1 receptor agonist. In some embodiments, the maintenance dose is between 15%-25% of the LTD for the GLP-1 receptor agonist.

The GLP-1 receptor agonist can comprise, for example, one or more of liraglutide, exenatide, lixisenatide, albiglutide, dulaglutide, semaglutide, taspoglutide, efpeglenatide, and tirzepatide.

In some embodiments, the GLP-1 receptor agonist comprises liraglutide, and the maintenance dose is between 65-85% of the LTD for liraglutide. In some embodiments, the GLP-1 receptor agonist comprises liraglutide, and the maintenance dose is between 40-60% of the LTD for liraglutide. In some embodiments, the GLP-1 receptor agonist comprises liraglutide, and the maintenance dose is between 15-35% of the LTD for liraglutide.

In some embodiments, the GLP-1 receptor agonist comprises exenatide, and the maintenance dose is between 65-85% of the LTD for exenatide. In some embodiments, the GLP-1 receptor agonist comprises exenatide, and the maintenance dose is between 40-60% of the LTD for exenatide. In some embodiments, the GLP-1 receptor agonist comprises exenatide, and the maintenance dose is between 15-35% of the LTD for exenatide.

In some embodiments, the GLP-1 receptor agonist comprises lixisenatide, and the maintenance dose is between 65-85% of the LTD for lixisenatide. In some embodiments, the GLP-1 receptor agonist comprises lixisenatide, and the maintenance dose is between 40-60% of the LTD for lixisenatide. In some embodiments, the GLP-1 receptor agonist comprises lixisenatide, and the maintenance dose is between 15-35% of the LTD for lixisenatide.

In some embodiments, the GLP-1 receptor agonist comprises albiglutide, and the maintenance dose is between 65-85% of the LTD for albiglutide. In some embodiments, the GLP-1 receptor agonist comprises albiglutide, and the maintenance dose is between 40-60% of the LTD for albiglutide. In some embodiments, the GLP-1 receptor agonist comprises albiglutide, and the maintenance dose is between 15-35% of the LTD for albiglutide.

In some embodiments, the GLP-1 receptor agonist comprises dulaglutide, and the maintenance dose is between 65-85% of the LTD for dulaglutide. In some embodiments, the GLP-1 receptor agonist comprises dulaglutide, and the maintenance dose is between 40-60% of the LTD for dulaglutide. In some embodiments, the GLP-1 receptor agonist comprises dulaglutide, and the maintenance dose is between 15-35% of the LTD for dulaglutide.

In some embodiments, the GLP-1 receptor agonist comprises semaglutide, and the maintenance dose is between 65-85% of the LTD for semaglutide. In some embodiments, the GLP-1 receptor agonist comprises semaglutide, and the maintenance dose is between 40-60% of the LTD for semaglutide. In some embodiments, the GLP-1 receptor agonist comprises semaglutide, and the maintenance dose is between 15-35% of the LTD for semaglutide.

In some embodiments, the GLP-1 receptor agonist comprises taspoglutide, and the maintenance dose is between 65-85% of the LTD for taspoglutide. In some embodiments, the GLP-1 receptor agonist comprises taspoglutide, and the maintenance dose is between 40-60% of the LTD for taspoglutide. In some embodiments, the GLP-1 receptor agonist comprises taspoglutide, and the maintenance dose is between 15-35% of the LTD for taspoglutide.

In some embodiments, the GLP-1 receptor agonist comprises efpeglenatide, and the maintenance dose is between 65-85% of the LTD for efpeglenatide. In some embodiments, the GLP-1 receptor agonist comprises efpeglenatide, and the maintenance dose is between 40-60% of the LTD for efpeglenatide. In some embodiments, the GLP-1 receptor agonist comprises efpeglenatide, and the maintenance dose is between 15-35% of the LTD for efpeglenatide.

In some embodiments, the GLP-1 receptor agonist comprises tirzepatide, and the maintenance dose is between 65-85% of the LTD for tirzepatide. In some embodiments, the GLP-1 receptor agonist comprises tirzepatide, and the maintenance dose is between 40-60% of the LTD for tirzepatide. In some embodiments, the GLP-1 receptor agonist comprises tirzepatide, and the maintenance dose is between 15-35% of the LTD for tirzepatide.

In some embodiments, providing the GLP-1 receptor agonist with the pharmaceutically or nutritionally acceptable carrier to the patient at the maintenance dose lower than the LTD to be administered during the first period is further provided to be administered using substantially the same dosing interval and treatment duration as recommended for the LTD.

In some embodiments, the maintenance dose is the lowest diabetes therapeutic dose (LDTD). In some embodiments, providing the GLP-1 receptor agonist to the patient comprises providing the GLP-1 receptor agonist to the patient in accordance with a dose escalation scheme including the maintenance dose. It is contemplated that dose escalation schemes can minimize side effects in some methods. In some embodiments, the maintenance dose is higher than the LDTD but lower than the LOTD.

In some embodiments, the obesity is associated with a disease or disorder, the method comprises preventing or treating the disease or disorder, and the disease or disorder comprises one or more of obesity, nonalcoholic fatty liver disease (NAFLD), non-alcoholic steatohepatitis (NASH), type 2 diabetes mellitus (T2DM), hypertension, dyslipidemia, osteoarthritis, kidney disease, sleep apnea, and polycystic ovarian syndrome (PCOS). For example, in some embodiments, weight loss associated with an obesity treatment method can prevent or reduce the risk of development of T2DM.

In some embodiments, the method can further comprise providing at least one additional therapeutically active agent to be administered during the first period. In some embodiments, the at least one additional therapeutically active agent comprises a biguanide, a dipeptidyl peptidase IV (DPP IV) inhibitor, and a sodium-glucose cotransporter-2 (SGLT-2) inhibitor.

In some embodiments, the DPP IV inhibitor is selected from sitagliptin, vildagliptin, saxagliptin, linagliptin, gemigliptin, anagliptin, teneligliptin, alogliptin, trelagliptin, omarigliptin, evogliptin, gosogliptin, dutogliptin, or the pharmaceutically acceptable salt or hydrate thereof.

In some embodiments, the biguanide is metformin or the pharmaceutically acceptable salt or hydrate thereof. In some embodiments, the biguanide is metformin hydrochloride. In some embodiments, the metformin is formulated for immediate release. In some embodiments, the metformin is formulated for slow release.

In some embodiments, the SGLT-2 inhibitor is selected from canagliflozin and dapagliflozin.

In some embodiments, the insulin modulating nutritional therapy protocol comprises a hormonal nutritional therapy (HNT) protocol. The HNT protocol can comprise a reduced insulin fasting (RIF) protocol and/or a reduced insulin nutrition (RIN) protocol.

Contemplated RIF protocols can comprise an intermittent fasting protocol. In some embodiments, the RIF protocol is One-Meal-A-Day (OMAD). In some embodiments, the RIF protocol is 15-hour fasting. In some embodiments, the RIF protocol is 18-hour fasting.

Contemplated RIN protocols can comprise a high protein and high natural fats diet.

In some embodiments, the GLP-1 receptor agonist with a pharmaceutically or nutritionally acceptable carrier is provided in the form of pill, tablet or capsule. In some embodiments, the composition GLP-1 receptor agonist with a pharmaceutically or nutritionally acceptable carrier is suitable for oral administration. In some embodiments, the composition GLP-1 receptor agonist with a pharmaceutically or nutritionally acceptable carrier is in the form of an injectable composition.

In an aspect, a method for treating or preventing obesity in a subject is provided, comprising providing a GLP-1 receptor agonist with a pharmaceutically or nutritionally acceptable carrier to the patient at a maintenance dose to be administered during a first period, wherein during a second period that overlaps with the first period, the subject is on an insulin modulating nutritional therapy protocol comprising at least one of a RIN and a RIF protocol.

In some embodiments, the RIF protocol comprises fasting at least 12 consecutive hours each day of the second period. In some embodiments, the RIF protocol comprises skipping at least one meal. In some embodiments, the at least 12 consecutive hours begins after breakfast and before at least one of lunch and dinner. In some embodiments, the at least 12 consecutive hours begins after lunch and before dinner. In some embodiments, the RIF protocol comprises fasting at least 16 consecutive hours each day of the second period. In some embodiments, the RIF protocol comprises fasting at least 20 consecutive hours each day of the second period. In some embodiments, the RIF protocol comprises fasting at least 23 consecutive hours each day of the second period. In some embodiments, the RIF protocol comprises an intermittent fasting protocol.

In some embodiments, the RIN protocol requires less than 25% of daily calories consumed by the subject to be from carbohydrates. In some embodiments, the RIN protocol requires less than 20% of daily calories consumed by the subject to be from carbohydrates. In some embodiments, the RIN protocol requires less than 15% of daily calories consumed by the subject to be from carbohydrates. In some embodiments, the RIN protocol requires less than 10% of daily calories consumed by the subject to be from carbohydrates.

In some embodiments, the maintenance dose is a dose lower than a LTD.

In some embodiments, the maintenance dose is between 10%-95% of the LTD for the GLP-1 receptor agonist.

In some embodiments, the maintenance dose is between 65%-95% of the LTD for the GLP-1 receptor agonist.

In some embodiments, the maintenance dose is between 70%-90% of the LTD for the GLP-1 receptor agonist.

In some embodiments, the maintenance dose is between 70%-80% of the LTD for the GLP-1 receptor agonist.

In some embodiments, the maintenance dose is between 35%-65% of the LTD for the GLP-1 receptor agonist.

In some embodiments, the maintenance dose is between 40%-60% of the LTD for the GLP-1 receptor agonist.

In some embodiments, the maintenance dose is between 45%-55% of the LTD for the GLP-1 receptor agonist.

In some embodiments, the maintenance dose is between 10%-35% of the LTD for the GLP-1 receptor agonist.

In some embodiments, the maintenance dose is between 15%-30% of the LTD for the GLP-1 receptor agonist.

In some embodiments, the maintenance dose is between 15%-25% of the LTD for the GLP-1 receptor agonist.

In some embodiments, the maintenance dose is the LDTD. In some embodiments, providing the GLP-1 receptor agonist to the patient comprises providing the GLP-1 receptor agonist to the patient in accordance with a dose escalation schedule including the maintenance dose. In some embodiments, the maintenance dose is higher than the LDTD but lower than the LOTD.

The GLP-1 receptor agonist can comprise, for example, one or more of liraglutide, exenatide, lixisenatide, albiglutide, dulaglutide, semaglutide, taspoglutide, efpeglenatide, and tirzepatide.

In some embodiments, the obesity is associated with a disease or disorder, the method comprises preventing or treating the disease or disorder and the disease or disorder comprises one or more of obesity, nonalcoholic fatty liver disease (NAFLD), non-alcoholic steatohepatitis (NASH), type 2 diabetes mellitus (T2DM), hypertension, dyslipidemia, osteoarthritis, kidney disease, sleep apnea, and polycystic ovarian syndrome (PCOS).

In some embodiments, the method can further comprise providing at least one additional therapeutically active agent to be administered during the first period. In some embodiments, the at least one additional therapeutically active agent comprises a biguanide, a dipeptidyl peptidase IV (DPP IV) inhibitor, and a sodium-glucose cotransporter-2 (SGLT-2) inhibitor.

In an aspect, a method for treating or preventing obesity in a subject is provided, comprising providing a GLP-1 receptor agonist with a pharmaceutically or nutritionally acceptable carrier to the patient at a maintenance dose to be administered during a first period, wherein during a second period that overlaps with the first period, the subject is on an insulin modulating nutritional therapy protocol comprising at least one of a RIN and a RIF protocol.

In some embodiments, the second period is at least two weeks. In some embodiments, the second period is at least three weeks. In some embodiments, the second period is at least four weeks. In some embodiments, the second period is at least five weeks. In some embodiments, the second period is at least six weeks. In some embodiments, the second period is at least one month. In some embodiments, the second period is at least three months. In some embodiments, the second period is at least six months.

In some embodiments, the first period is entirely within the second period. In some embodiments, the first period and the second period overlap at least one week. In some embodiments, the first period and the second period overlap at least two weeks. In some embodiments, the first period and the second period overlap at least three weeks. In some embodiments, the first period and the second period overlap at least one month. In some embodiments, the first period and the second period overlap at least two months. In some embodiments, the first period and the second period overlap at least three months.

In some embodiments, the first period is at least one week. In some embodiments, the first period is at least two weeks. In some embodiments, the first period is at least three weeks. In some embodiments, the first period is at least four weeks. In some embodiments, the first period is at least five weeks. In some embodiments, the first period is at least six weeks. In some embodiments, the first period is at least one month. In some embodiments, the first period is at least three months. In some embodiments, the first period is at least six months.

In some embodiments, the treatment results in an improvement, slowing the progression of, or delaying a metabolic disorder such as nonalcoholic fatty liver disease (NAFLD), non-alcoholic steatohepatitis (NASH), type 2 diabetes mellitus (T2DM), hypertension, dyslipidemia, osteoarthritis, kidney disease, sleep apnea, polycystic ovarian syndrome (PCOS), and complications associated therewith.

In some embodiments, the treatment results in an improvement, slowing the progression of, or delaying a metabolic disorder that is greater than that obtained with the lowest therapeutic dose (LTD) dose of the GLP-1 receptor agonist without HNT. In some embodiments, the treatment results in greater long term tolerability and reduced risk of side effects when compared to treatment with the LTD dose of the GLP-1 receptor agonist. In some embodiments, the treatment is the initial or first-line treatment of diabetes. In some embodiments, the subject is not receiving any diabetes therapy prior to treatment.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows an example fasting model;

FIG. 2 shows certain GLP-1 receptor agonists approved for the treatment of type II diabetes, and certain GLP-1 receptor agonists approved (or in development) for weight management, along with the LOTDs and LDTDs;

FIG. 3 is an informational chart for GLP-1 receptor agonists approved for weight management;

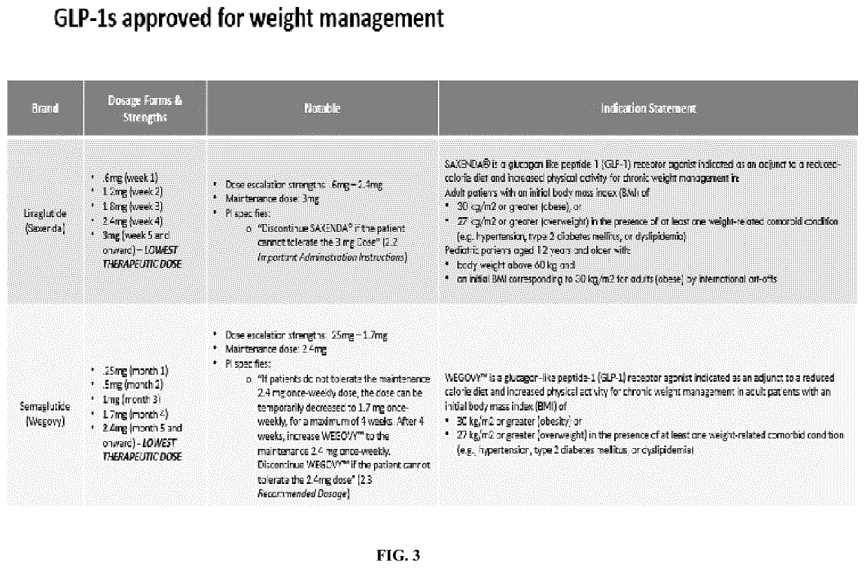

FIG. 4 is an informational chart for GLP-1 based therapies in development for weight management;

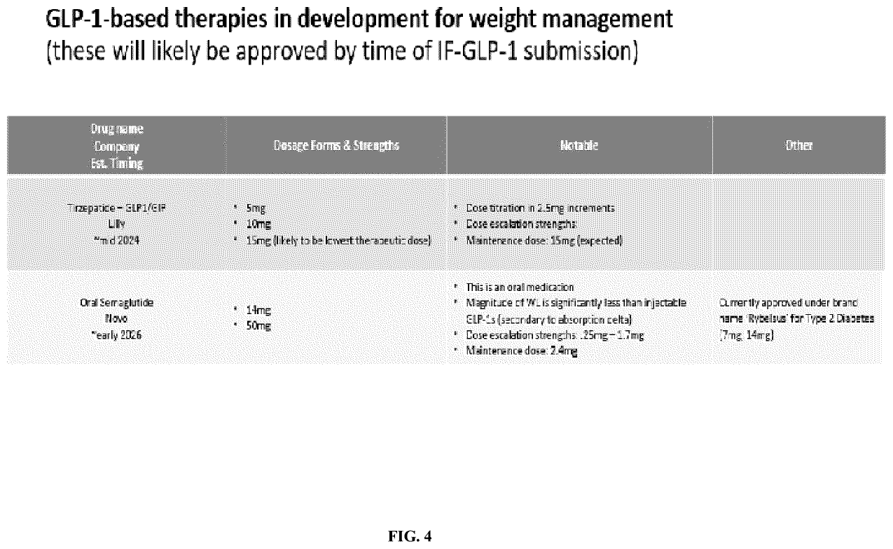

FIG. 5 shows dosing schedule information for Saxenda® available at www.saxenda.com;

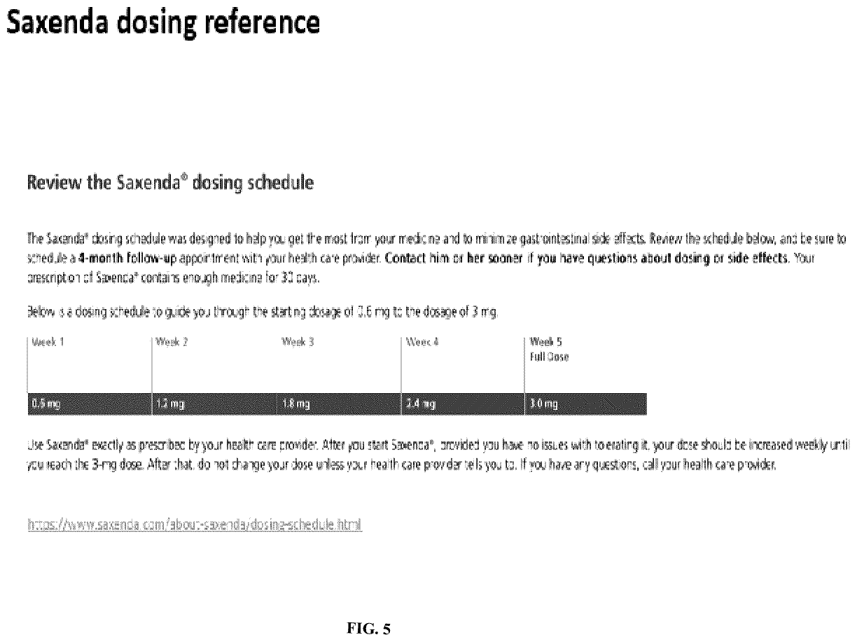

FIG. 6 shows dosing schedule information for Wegovy™ available at www.wegovy.com:

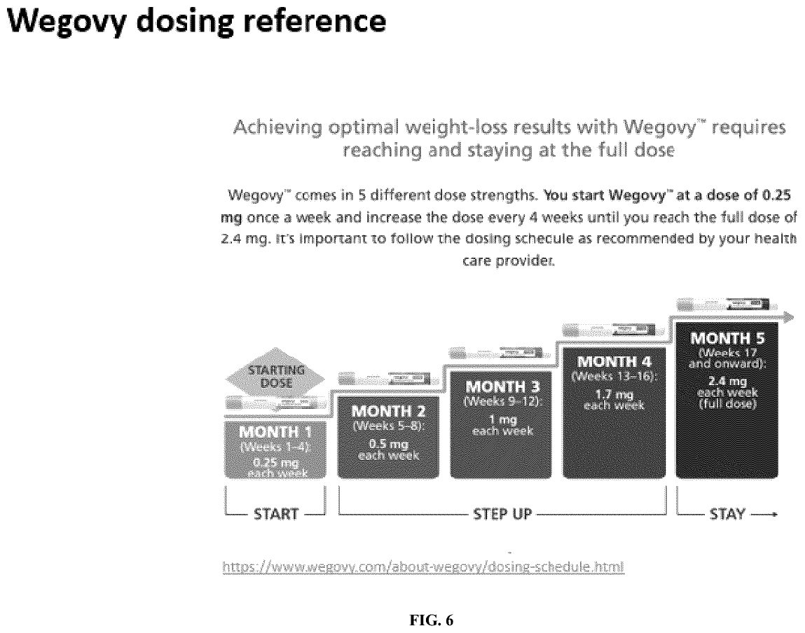

FIG. 7 shows dosing information for Wegovy™ available at www.novomedlink.com; and

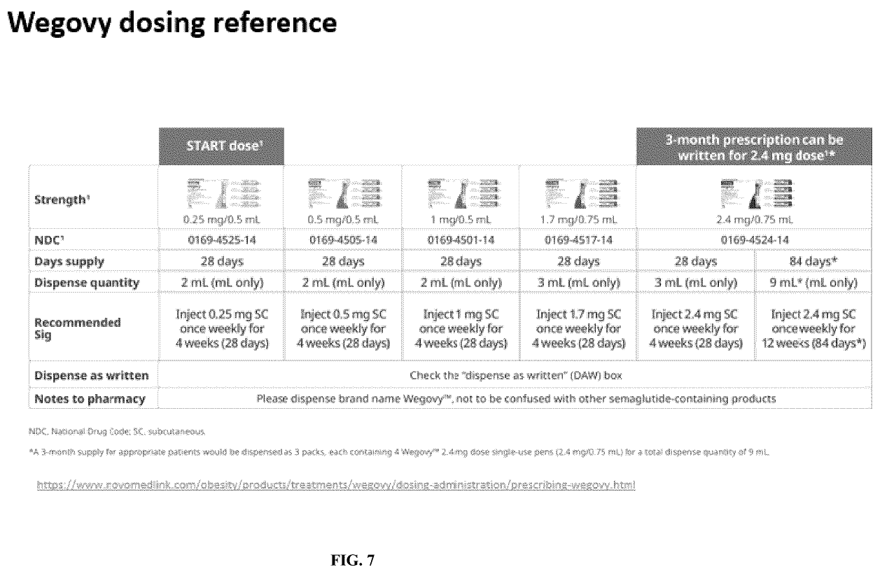

FIG. 8 shows dosing information for Wegovy™ available at www.novomedlink.com, which indicates Wegovy™ should be discontinued if the patient cannot tolerate the 2.4 mg dose.

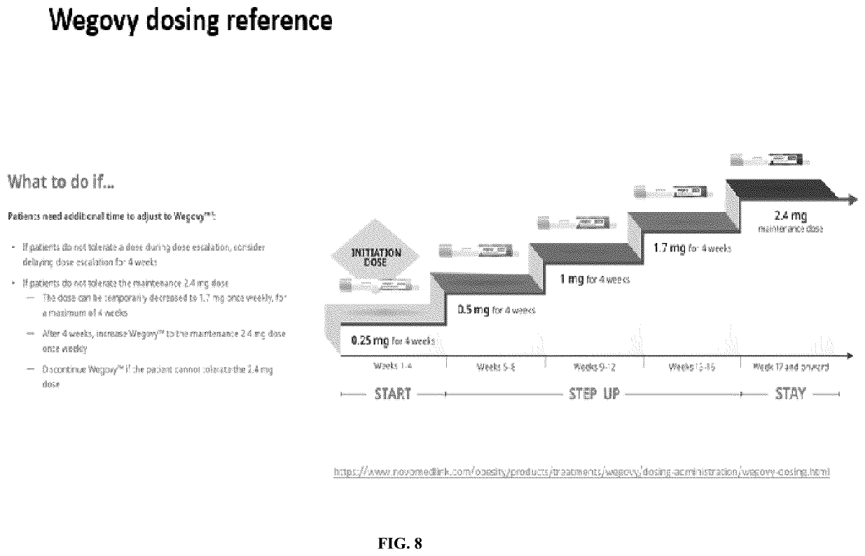

### DETAILED DESCRIPTION OF THE DISCLOSURE

Obesity is a surging epidemic across the world, in particularly in the United States. In 2017, the US Center for Disease Control and Prevention estimated that 42% of U.S. adults are obese, which represents “the highest incidence ever recorded in America.” See American Society for Metabolic & Bariatric Surgery. Obesity in America. https://asmbs.org/app/uploads/2021/07/Obesity-in-America-Fact-Sheet-2021.pdf). From a pathophysiological perspective, obesity results in significant metabolic derangement and has been found to induce a state of chronic inflammation. As a result, obesity is directly connected with over 40 diseases and is a leading cause of mortality (both preventable and premature). During the recent Covid-19 pandemic, obesity was found to be a leading cause of mortality and carried three times the risk for Covid-related hospitalization. See Hussain A. Mahawar K. Xia Z. Yang W. El-Hasani S. Obesity and mortality of COVID-19. Meta-analysis. Obes Res Clin Pract. 2020 July-August; 14 (4): 295-300. doi: 10.1016/j.orcp.2020.07.002. Epub 2020 Jul. 9; Center for Disease Control. Obesity Worsens Outcomes from COVID-19. https://www.cdc.gov/obesity/data/obesity-and-covid-19.html.

The manifold physiological consequences associated with obesity has produced a commensurate economic burden to individuals and to society writ large. A recent study noted that the aggregate health care costs to the United States in 2016 was over $260 billion dollars. See John Cawley, PhD; Adam Biener, PhD; Chad Meyerhoefer, PhD; Yuchen Ding. PhD; Tracy Zvenyach, PhD. NP. B Gabriel Smolarz, MD, MS; and Abhilasha Ramasamy, MS. Direct Medical Costs Of Obesity In The United States And The Most Populous States. J Manag Care Spec Pharm. 2021; 27 (3):354-66.

Addressing weight loss is therefore highly warranted given the rising rates of obesity. the association of multitude of diseases associated, and the precipitous economic burden.

Approved drugs in the field are developed and approved based upon US Food and Drug Administration regulations and do not require nor appreciate the benefits of hormonal nutritional therapy (HNT). The dietary component in obesity trials, if any, is merely caloric restriction without any focus on timing, frequency or food composition.

Applicant surprisingly discovered that a combination of HNT and hormonal medicinal therapy (HMT) can have a synergistic effect in treating and preventing obesity and its related diseases and disorders. Accordingly, some methods of the disclosure can treat or prevent obesity and its related diseases and disorders by stimulating rapid and sustained fat release via two distinct and synergistic steps, namely HNT and HMT. As used herein, HNT can be viewed as purposeful nutritional manipulation to achieve desired hormonal responses and biological effects within the body. HNT can comprise one or both of what type of food composition is ingested and the timing and frequency of food ingestion. As used herein, HMT can be viewed as the utilization of biopharmaceuticals, for example, a GLP-1 receptor agonist, which can advantageously be administered in an amount lower than the lowest therapeutic dose (LTD) of the GLP-1 receptor agonist.

### Hormonal Nutritional Therapy (HNT)

HNT targets physiological levers to induce a given metabolic response. HNT can have two sub-components: Reduced Insulin Fasting (RIF) and Reduced Insulin Nutrition (RIN). Multiple hormones are critical in modulating weight loss and preventing the sequelae of associated health problems. In some embodiments, the methods described herein focus on modulating physiology such that weight loss can be accomplished. This fasting component of a RIF protocol can engage a physiological response resulting in an enhanced metabolic and neuronal physiological environment conducive to weight loss. The RIN protocol can focus on further magnifying this metabolic state by focusing on food intake that does not promote the secretion of insulin.

In some contemplated methods, these two elements of HNT are combined to target metabolic hormones by optimizing timing and meal composition. The result is a highly effective means of targeting and modulating the metabolic system, thus creating physiological milieu in which fat is release from cells and burned for energy with the resultant induction of weight loss.

### 1. Reduced Insulin Fasting (RIF)

Intermittent fasting has gained notoriety as a means of reducing weight and improving overall health. See Albosta, M., Bakke, J. Intermittent fasting: is there a role in the treatment of diabetes? A review of the literature and guide for primary care physicians. Clin Diabetes Endocrinol 7, 3 (2021). https://doi.org/10.1186/s40842-020-00116-1. The term intermittent fasting (IF) is used as an umbrella term that describes a wide variety of cating cycle approaches. In the context of HNT, the term Reduced-Insulin Fasting (RIF) is the term applied. The basic tenet of RIF involves modulating blocks of time for eating and non-eating, therefore RIF fits into the IF schema of programs.

Insulin is a key target of HNT as it plays a pivotal role in terms of weight gain. Insulin is a gateway hormone that directs storage vs. use of energy (sugar). Higher levels of circulating insulin induces more storage of energy into fat stores. High insulin levels can also paradoxically elicit an increased sense of hunger. See Rezek M. The role of insulin in the glucostatic control of food intake. Can J Physiol Pharmacol. 1976 October; 54 (5): 650-65. doi: 10.1139/y76-091. PMID: 825207.

RIF programs are therefore targeted at reducing circulating insulin levels by activating fasting-induced metabolic switches. There are various IF approaches to effectively modulate this desired metabolic effect, including, for example:
Time-restricted eating. Involves fasting every day for 12 hours or longer and eating in the remaining hours. A popular example is the 16/8 method. It features a daily 16-hour fast and an 8-hour eating window wherein you can fit in 2, 3, or more meals.
The 5:2 method. The 5:2 diet involves eating as you normally do 5 days of the week and restricting your calorie intake to 500-600 on the remaining 2 days.
The 16:8 method. Sixteen fasting hours cycled by 8 non-fasting hours.
Eat Stop Eat. Eat Stop Eat involves a 24-hour fast once or twice per week.
Alternate-day fasting. With alternate-day fasting, the goal is to fast every other day.
The Warrior Diet. The Warrior Diet was among the first popular diets to include a form of intermittent fasting. It involves eating small amounts of raw fruits and vegetables during the day and eating one large meal at night.

See e.g., Snyder, C., Gunnars. K., Tinsley, G. Pros and Cons of 5 Intermittent Fasting Methods. Healthline.com. https://www.healthline.com/nutrition/6-ways-to-do-intermittent-fasting.

The above programs, among others, fit underneath the larger IF umbrella and thus comport with the RIF component of HNT. These programs could reduce circulating insulin levels. This claim is supported by a vast arc of published data. See e.g., Welton S, Minty R. O'Driscoll T, Willms H, Poirier D, Madden S, Kelly L. Intermittent fasting and weight loss: Systematic review. Can Fam Physician. 2020 February; 66 (2): 117-125. PMID: 32060194; PMCID: PMC7021351. The RIF protocol can vary for individuals based on, for example, an individual's lifestyle and schedule, eating patterns, ability to withstand hunger, etc.

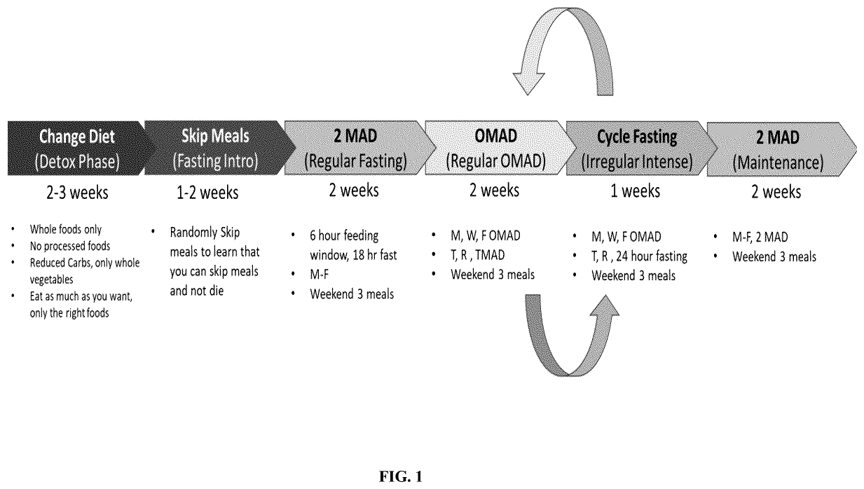

An example fasting model is depicted in FIG. 1. This graphic depicts the rate at which a patient can move along the journey if they are able to successfully navigate it. The journey can be a slow gradual process as the patient and their biology adapts to a new nutritional model.

### 2. Reduced Insulin Nutrition (RIN)

Endogenous insulin levels are naturally raised by ingestion of food. Particular foods stimulate more insulin production. In terms of macronutrients, the following elements frame the reduced insulin nutritional plan:
Carbohydrates (especially refined carbohydrates) induces the greatest secretion of insulin;
Proteins induces less insulin secretion than carbohydrates (not a major contributor to hyperinsulinemia);
Dietary fat does not raise endogenous insulin.

See Fung, Jason. What and when to eat to reduce insulin. https://www.dietdoctor.com/what-and-when-to-eat-to-reduce-insulin.

While foods contain a mix of macronutrients, RIN focuses on ingestion of foods that are on the lower end of the insulin-induction spectrum. As such, this can involve eating foods that are high in protein and natural fats while avoiding foods that are high in carbohydrates/sugar, refined grains, and processed food in general. In some aspects, the macronutrient composition can be a primary focus with a target of consuming between 20-60 grams of carbohydrates a day. While that can be a focus, it is much more complex as carbohydrates are not the only substance that cause insulin release. Non-caloric sweeteners also cause insulin release. Insulin release due to protein is variable based upon different conditions. Regardless of the precise method, the goal of RIN is precisely that, to reduce insulin release by nutritional methods.

### Hormonal Medicinal Therapy (HMT)

HMT can comprise the utilization of biopharmaceuticals, optionally in low-doses, to augment metabolic reprogramming and induce weight loss (independently and/or complementarily with HNT). The biopharmaceuticals can comprise gut-brain peptides that target the actions of glucagon-like peptide-1 (GLP-1) either through receptor agonism or use of GLP-1 synthetic peptides (e.g., exendin-4). Additionally, the GLP-1 receptor agonist (e.g., low-dose GLP-1 receptor agonist) can also be used in combination with other peptides, such as DPP IV inhibitors, metformin, or SGLT-2 inhibitors to elicit a desired metabolic effect.

Targeting GLP-1 as means of reducing body weight has been proven through the use of GLP-1 like therapeutics for the treatment of Type 2 Diabetes. Recently, the use of GLP-1 receptor agonism has been approved by the FDA specifically as weight loss products in patients with clinical obesity.

In the context of HNT, low-dose GLP-1 receptor agonism addresses another significant gap. Hunger is tied to the release of specific neuro-hormones the function in cadence with typical mealtimes. The difficulty for many people who attempt fasting-based programs is that these feeding hormones continue to be produced and thus hunger is a common associated complaint that often leads to non-compliance. Exogenous GLP-1 can be a complimentary solution to this issue as even low-doses can have a significant impact on satiety.

### 1. The GLP-1 Paradox

The weight loss potential and the associated benefits of using GLP-1-based therapies have been well-characterized. Specifically, GLP-1 is expressed in the L-cells of the intestine, the neurons located in the caudal brainstem and hypothalamus, and as well as the alpha-cells of the pancreas. The pharmacological effects specific to weight loss induction include decreased food intake and delays in gastric emptying and gut motility. GLP-1 also exerts a central effect of regulating feeding. See Shah, M., Vella, A. “Effects of GLP-1 on appetite and weight.” Rev Endocr Metab Disord. 2014 September: 15 (3): 181-187.

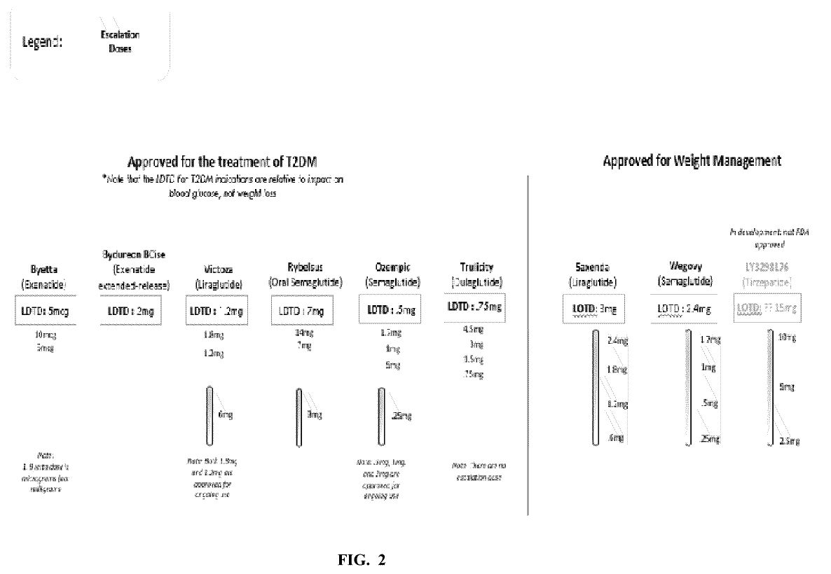

The magnitude of weight loss associated with GLP-1 therapies has been proven to be highly dose dependent. Therefore, the dosing regimens approved by the US Food and Drug Administration for lowering blood sugar in patients with Type 2 Diabetes have been abandoned by manufactures in favor of significantly higher doses for use in the context of weight loss in obese patients. FIG. 2 shows certain GLP-1 receptor agonists approved for the treatment of type II diabetes, and certain GLP-1 receptor agonists approved (or in development) for weight management, along with certain dosing information.

Paradoxically, the tolerability associated with GLP-1 therapy is inversely related to dose strength. As the dose of GLP-1 is increased, tolerability to the therapies decrease. Associated with these escalating GLP-1 dosing regimens is an increase in the cost-of-goods (COGS) to manufacture obesity medications. These COGS are secondarily reflected in likewise escalating pricing for the payer and the patient.

### 2. HMT-Low Dose GLP-1 Receptor Agonists (and Combinations Thereof)

The HMT approach of some contemplated methods of the disclosure uniquely focus on doses of GLP-1 that are lower than what is currently approved and indicated for weight loss in patients with obesity. In some aspects, the lower-than-approved doses of GLP-1, for example, in combination with HNT, can exert and effect that complements and augments the magnitude of weight loss achieved. Additionally, low-dose GLP-1 can be a therapeutic compliment to solve for the appetite issue that is associated with fasting-based programs.

The advantageous of low-dose medicinal use of GLP-1 receptor agonists with HNT are manifold, and can include, enhanced tolerability, reduction of other side-effects associated with escalating doses of GLP-1 receptor agonists, solving for the issue of hunger associated with fasting-based programs, optimized manufacturing and COGS, resulting in lower cost to payer and consumer, and the potential to greatly enhance overall patient health by harnessing the independent benefits of HNT with those of HMT.

### HNT+HMT

The distinct physiological benefits associated with HNT/HMT is proposed here to have profound weight loss effects. Weight loss is independently associated with a wide range of significant health benefits; however, the methods described herein can elicit independent benefits given that both the RIF component of HNT, and the direct therapeutic benefits of low-dose GLP-1 have direct benefits independent of weight loss. These independent benefits are directly tied to well-characterized health benefits for seen in non-obesity related diseases. See Sutton. E., Beyl. R., et al. “Early Time-Restricted Feeding Improves Insulin Sensitivity, Blood Pressure, and Oxidative Stress Even without Weight Loss in Men with Prediabetes.” Cell Metabolism, Volume 27, Issue 6, 5 Jun. 2018, Pages 1212-1221.

The methods described herein can advantageously treat or prevent obesity, as well as diseases and disorders associated with obesity, including, for example, nonalcoholic fatty liver disease (NAFLD), non-alcoholic steatohepatitis (NASH), type 2 diabetes mellitus (T2DM), hypertension, dyslipidemia, osteoarthritis, kidney disease, sleep apnea, and polycystic ovarian syndrome (PCOS).

Additionally, the methods described herein can advantageously solve major issues linked to compliance. For example, the difficulty associated with hunger-related fasting programs can be solved through low-dose GLP-1 receptor agonists. As another example, the discontinuation rates associated with approved anti-obesity agents can be resolved by using doses of GLP-1 receptor agonists that are lower than the FDA-approved doses for weight loss.

By optimizing the health benefits of composition and timing of food intake combined with low-dose therapeutics, significant changes in personal health, impacting both macro and micro (consumer) healthcare costs can be achieved.

### Certain Terminology

As used herein and in the appended claims, the singular forms “a.” “and,” and “the” include plural referents unless the context clearly dictates otherwise. Thus, for example, reference to “an agent” includes a plurality of such agents, and reference to “the composition” includes reference to one or more compositions (or to a plurality of compositions) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus in some embodiments, the number or numerical range varies between 1% and 10% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### Definitions

As used in the specification and appended claims, unless specified to the contrary, the following terms have the meaning indicated below.

“Pharmaceutically acceptable salt” as used herein includes both acid and base addition salts. In some embodiments, the pharmaceutically acceptable salt of any one of the compounds described herein is the form approved for use by the US Food and Drug Administration. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid. malonic acid, succinic acid, fumaric acid, tartaric acid, citric acid, benzoic acid, cinnamic acid, mandelic acid, methanesulfonic acid, ethanesulfonic acid. p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates. propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (sec. for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine. 2-dimethylaminoethanol. 2-diethylaminoethanol, dicyclohexylamine, lysine, arginine, histidine, caffeine. procaine. N,N-dibenzylethylenediamine, chloroprocaine, hydrabamine, choline, betaine, ethylenediamine, ethylenedianiline. N-methylglucamine. glucosamine, methylglucamine, theobromine, purines, piperazine, piperidine. N-ethylpiperidine. polyamine resins and the like. See Berge et al., supra.

As used herein. “hydrates” are compounds that contain either stoichiometric or non-stoichiometric amounts of water, and, in some embodiments, are formed during the process of crystallization with water. Hydrates are meant to include the hydrates of any one of the compounds described herein that is approved for use by the US Food and Drug Administration.

The term “acceptable” with respect to a formulation, composition or ingredient, as used herein. means having no persistent detrimental effect on the general health of the subject being treated.

The terms “administer.” “administered.” “administering.” “administration.” and the like, as used herein, refer to the methods that may be used to enable delivery of compounds or compositions to the desired site of biological action. These methods include, but are not limited to, oral routes, intraduodenal routes, parenteral injection (including intravenous, subcutaneous, intraperitoneal, intramuscular, intravascular or infusion), topical, and rectal administration. Those of skill in the art are familiar with administration techniques that can be employed with the compounds and methods described herein. In some embodiments, the compounds and compositions described herein are administered orally. In some embodiments, the compounds and compositions described herein are administered by injection.

The term “subject.” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating” or “palliating” or “ameliorating” are used interchangeably herein. These terms refer to an approach for obtaining beneficial or desired results including but not limited to anti-obesity effect, weight loss effect, therapeutic benefit and/or a prophylactic benefit. By “therapeutic benefit” or “anti-obesity effect” is meant eradication or amelioration of the underlying disorder being treated. A therapeutic benefit is achieved with the eradication or amelioration of one or more of the physiological symptoms associated with the underlying disorder (e.g., an improvement in: hyperglycemia, polyuria, polydipsia, polyphagia, diabetic dermadromes, excess weight, etc.) such that an improvement is observed in the patient, notwithstanding that the patient may still be afflicted with the underlying disorder. Also, a therapeutic benefit is achieved with the eradication or amelioration of one or more of the complications associated with the underlying disorder (e.g., cardiovascular disease). For prophylactic benefit, the compositions may be administered to a patient at risk of developing a particular disease, or to a patient reporting one or more of the physiological symptoms of a disease, even though a diagnosis of this disease may not have been made.

The terms “diabetes” and “diabetes mellitus” are used interchangeably herein. These terms refers to type 1 diabetes mellitus, type 2 diabetes mellitus, complications of diabetes mellitus, and of neighboring disease states. As used herein, diabetes or diabetes mellitus (DM) refers to a group of metabolic disorders in which there are high blood sugar levels over a prolonged period.

As used herein, the term “maintenance dose” refers to (i) a dose of a GLP-1 receptor agonist where there is no dose escalation scheme, or (ii) a final maintenance dose of a dose escalation scheme.

### Methods

Provided herein are methods for treating or preventing obesity and/or a disease or disorder associated with obesity in a subject, comprising providing a glucagon-like peptide-1 (GLP-1) receptor agonist with a pharmaceutically or nutritionally acceptable carrier to the patient at a maintenance dose to be administered during a first period, wherein during a second period that overlaps with the first period, the subject is on an insulin modulating nutritional therapy protocol. In some embodiments, the maintenance dose is lower than a lowest therapeutic dose (LTD). In some embodiments, the insulin modulating nutritional therapy protocol comprises one or both of RIF and RIN.

### Lowest Therapeutic Dose

As used herein, the lowest therapeutic dose (LTD) of a GLP-1 receptor agonist refers to the lowest therapeutic dose in a specific dosage form (injectable, oral, or extended release) approved by the US Food and Drug Administration and is not marked as “discontinued” by the Orange Book database (world-wide web at address accessdata.fda.gov/scripts/cder/ob/) as of the filing date of this application. If the GLP-1 receptor agonist has been approved for weight management or obesity by the US Food and Drug Administration and is not marked as “discontinued” by the Orange Book database (world-wide web at address accessdata.fda.gov/scripts/cder/ob/) as of the filing date of this application, the LTD of the GLP-1 receptor agonist is the lowest obesity therapeutic dose (LOTD) in a specific dosage form (injectable, oral, or extended release) approved by the US Food and Drug Administration. If the GLP-1 receptor agonist has not been approved for weight management or obesity by the US Food and Drug Administration as of the filing date of this application and has been approved for diabetes as of the filing date of this application and is not marked as “discontinued” by the Orange Book database (world-wide web at address accessdata.fda.gov/scripts/cder/ob/) as of the filing date of this application, the LTD of the GLP-1 receptor agonist is the lowest diabetes therapeutic dose (LDTD) in a specific dosage form (injectable, oral, or extended release) approved by the US Food and Drug Administration. Where the US Food and Drug Administration approves dose escalation for the approved single agent for weight management and/or diabetes treatment, the LTD, LOTD and/or LDTD refer to the maintenance dose. The lowest diabetes/obesity therapeutic dose does not include the dose as recommended by a physician for cases wherein the lowest diabetes/obesity therapeutic dose is not the same dose as recommended by the US Food and Drug Administration. Further, the lowest diabetes/obesity dose of the GLP-1 receptor agonists described herein refers to the dose of the form of GLP-1 receptor agonists approved for use by the US Food and Drug Administration, and any pharmaceutically acceptable salts thereof.

Table 1 shows the LTD for several GLP-1 receptor agonists approved for use for diabetes and/or weight management.

TABLE 1

| Brand Name | GLP-1 RA | Doses | | LTD | |
|---|---|---|---|---|---|
| Byetta | Exenatide | 5 | mcg | 5 | mcg |
| | | 10 | mcg | | |
| Bydureon | Exenatide | 2 | mg | 2 | mg |
| BCise | (extended release) | | | | |
| Rybelsus | Semaglutide (oral) | 7 | mg | 7 | mg |
| | | 14 | mg | | |
| | | (escalation dosing | | | |
| | | starting with 3 mg | | | |
| | | initiating dose) | | | |
| Trulicity | Dulaglutide | .75 | mg | .75 | mg |
| | | 1.5 | mg | | |
| | | 3 | mg | | |
| | | 4.5 | mg | | |
| Saxenda | Liraglutide | 3 | mg | 3 | mg |
| | | (escalation dosing | | | |
| | | between .6 mg and 2.4 | | | |
| | | mg before maintenance | | | |
| | | dose of 3 mg) | | | |
| Wegovy | Semaglutide | 2.4 | mg | 2.4 | mg |

(escalation dosing
between .25 mg and 1.7
mg before maintenance
dose of 3 mg)

In some embodiments, the maintenance dose is between 10%-95% of the LTD for the GLP-1 receptor agonist.

In some embodiments, the maintenance dose is between 65%-95% of the LTD for the GLP-1 receptor agonist.

In some embodiments, the maintenance dose is between 70%-90% of the LTD for the GLP-1 receptor agonist.

In some embodiments, the maintenance dose is between 70%-80% of the LTD for the GLP-1 receptor agonist.

In some embodiments, the maintenance dose is between 35%-65% of the LTD for the GLP-1 receptor agonist.

In some embodiments, the maintenance dose is between 40%-60% of the LTD for the GLP-1 receptor agonist.

In some embodiments, the maintenance dose is between 45%-55% of the LTD for the GLP-1 receptor agonist.

In some embodiments, the maintenance dose is between 10%-35% of the LTD for the GLP-1 receptor agonist.

In some embodiments, the maintenance dose is between 15%-30% of the LTD for the GLP-1 receptor agonist.

In some embodiments, the maintenance dose is between 15%-25% of the LTD for the GLP-1 receptor agonist.

The GLP-1 receptor agonist can comprise, for example, one or more of liraglutide, exenatide, lixisenatide, albiglutide, dulaglutide, semaglutide, taspoglutide, efpeglenatide, and tirzepatide.

In some embodiments, the GLP-1 receptor agonist comprises liraglutide, and the maintenance dose is between 65-85% of the LTD for liraglutide. In some embodiments, the GLP-1 receptor agonist comprises liraglutide, and the maintenance dose is between 40-60% of the LTD for liraglutide. In some embodiments, the GLP-1 receptor agonist comprises liraglutide, and the maintenance dose is between 15-35% of the LTD for liraglutide.

In some embodiments, the GLP-1 receptor agonist comprises exenatide, and the maintenance dose is between 65-85% of the LTD for exenatide. In some embodiments, the GLP-1 receptor agonist comprises exenatide, and the maintenance dose is between 40-60% of the LTD for exenatide. In some embodiments, the GLP-1 receptor agonist comprises exenatide, and the maintenance dose is between 15-35% of the LTD for exenatide.

In some embodiments, the GLP-1 receptor agonist comprises lixisenatide, and the maintenance dose is between 65-85% of the LTD for lixisenatide. In some embodiments, the GLP-1 receptor agonist comprises lixisenatide, and the maintenance dose is between 40-60% of the LTD for lixisenatide. In some embodiments, the GLP-1 receptor agonist comprises lixisenatide, and the maintenance dose is between 15-35% of the LTD for lixisenatide.

In some embodiments, the GLP-1 receptor agonist comprises albiglutide, and the maintenance dose is between 65-85% of the LTD for albiglutide. In some embodiments, the GLP-1 receptor agonist comprises albiglutide, and the maintenance dose is between 40-60% of the LTD for albiglutide. In some embodiments, the GLP-1 receptor agonist comprises albiglutide, and the maintenance dose is between 15-35% of the LTD for albiglutide.

In some embodiments, the GLP-1 receptor agonist comprises dulaglutide, and the maintenance dose is between 65-85% of the LTD for dulaglutide. In some embodiments, the GLP-1 receptor agonist comprises dulaglutide, and the maintenance dose is between 40-60% of the LTD for dulaglutide. In some embodiments, the GLP-1 receptor agonist comprises dulaglutide, and the maintenance dose is between 15-35% of the LTD for dulaglutide.

In some embodiments, the GLP-1 receptor agonist comprises semaglutide, and the maintenance dose is between 65-85% of the LTD for semaglutide. In some embodiments, the GLP-1 receptor agonist comprises semaglutide, and the maintenance dose is between 40-60% of the LTD for semaglutide. In some embodiments, the GLP-1 receptor agonist comprises semaglutide, and the maintenance dose is between 15-35% of the LTD for semaglutide.

In some embodiments, the GLP-1 receptor agonist comprises taspoglutide, and the maintenance dose is between 65-85% of the LTD for taspoglutide. In some embodiments, the GLP-1 receptor agonist comprises taspoglutide, and the maintenance dose is between 40-60% of the LTD for taspoglutide. In some embodiments, the GLP-1 receptor agonist comprises taspoglutide, and the maintenance dose is between 15-35% of the LTD for taspoglutide.

In some embodiments, the GLP-1 receptor agonist comprises efpeglenatide, and the maintenance dose is between 65-85% of the LTD for efpeglenatide. In some embodiments, the GLP-1 receptor agonist comprises efpeglenatide, and the maintenance dose is between 40-60% of the LTD for efpeglenatide. In some embodiments, the GLP-1 receptor agonist comprises efpeglenatide, and the maintenance dose is between 15-35% of the LTD for efpeglenatide.

In some embodiments, the GLP-1 receptor agonist comprises tirzepatide, and the maintenance dose is between 65-85% of the LTD for tirzepatide. In some embodiments, the GLP-1 receptor agonist comprises tirzepatide, and the maintenance dose is between 40-60% of the LTD for tirzepatide. In some embodiments, the GLP-1 receptor agonist comprises tirzepatide, and the maintenance dose is between 15-35% of the LTD for tirzepatide.

In some embodiments, providing the GLP-1 receptor agonist with the pharmaceutically or nutritionally acceptable carrier to the patient at the maintenance dose lower than the LTD to be administered during the first period is further provided to be administered using substantially the same dosing interval and treatment duration as recommended for the LTD.

In some embodiments, the maintenance dose is the lowest diabetes therapeutic dose (LDTD). In some embodiments, providing the GLP-1 receptor agonist to the patient comprises providing the GLP-1 receptor agonist to the patient in accordance with a dose escalation schedule including the maintenance dose. In some embodiments, the maintenance dose is higher than the LDTD but lower than the LOTD.

In some embodiments, the disease or disorder comprises one or more of obesity, nonalcoholic fatty liver disease (NAFLD), non-alcoholic steatohepatitis (NASH), type 2 diabetes mellitus (T2DM), hypertension, dyslipidemia, osteoarthritis, kidney disease, sleep apnea, and polycystic ovarian syndrome (PCOS).

In some embodiments, the method can further comprise providing at least one additional therapeutically active agent to be administered during the first period. In some embodiments, the at least one additional therapeutically active agent comprises a biguanide, a dipeptidyl peptidase IV (DPP IV) inhibitor, and a sodium-glucose cotransporter-2 (SGLT-2) inhibitor.

In some embodiments, the DPP IV inhibitor is selected from sitagliptin, vildagliptin, saxagliptin, linagliptin, gemigliptin, anagliptin, teneligliptin, alogliptin, trelagliptin, omarigliptin, evogliptin, gosogliptin, dutogliptin, or the pharmaceutically acceptable salt or hydrate thereof.

In some embodiments, the biguanide is metformin or the pharmaceutically acceptable salt or hydrate thereof. In some embodiments, the biguanide is metformin hydrochloride. In some embodiments, the metformin is formulated for immediate release. In some embodiments, the metformin is formulated for slow release.

In some embodiments, the SGLT-2 inhibitor is selected from canagliflozin and dapagliflozin.

In some embodiments, the insulin modulating nutritional therapy protocol comprises a hormonal nutritional therapy (HNT) protocol. The HNT protocol can comprise a reduced insulin fasting (RIF) protocol and/or a reduced insulin nutrition (RIN) protocol.

In some embodiments, the RIF protocol comprises fasting at least 12 consecutive hours each day of the second period. In some embodiments, the RIF protocol comprises skipping at least one meal. In some embodiments, the at least 12 consecutive hours begins after breakfast and before at least one of lunch and dinner. In some embodiments, the at least 12 consecutive hours begins after lunch and before dinner. In some embodiments, the RIF protocol comprises fasting at least 16 consecutive hours each day of the second period. In some embodiments, the RIF protocol comprises fasting at least 20 consecutive hours each day of the second period. In some embodiments, the RIF protocol comprises fasting at least 23 consecutive hours each day of the second period. In some embodiments, the RIF protocol comprises an intermittent fasting protocol.

In some embodiments, the RIN protocol requires less than 25% of daily calories consumed by the subject to be from carbohydrates. In some embodiments, the RIN protocol requires less than 20% of daily calories consumed by the subject to be from carbohydrates. In some embodiments, the RIN protocol requires less than 15% of daily calories consumed by the subject to be from carbohydrates. In some embodiments, the RIN protocol requires less than 10% of daily calories consumed by the subject to be from carbohydrates.

In some embodiments, the second period is at least two weeks. In some embodiments, the second period is at least three weeks. In some embodiments, the second period is at least four weeks. In some embodiments, the second period is at least five weeks. In some embodiments, the second period is at least six weeks. In some embodiments, the second period is at least one month. In some embodiments, the second period is at least three months. In some embodiments, the second period is at least six months.

In some embodiments, the first period is entirely within the second period. In some embodiments, the first period and the second period overlap at least one week. In some embodiments, the first period and the second period overlap at least two weeks. In some embodiments, the first period and the second period overlap at least three weeks. In some embodiments, the first period and the second period overlap at least one month. In some embodiments, the first period and the second period overlap at least two months. In some embodiments, the first period and the second period overlap at least three months.

In some embodiments, the first period is at least one week. In some embodiments, the first period is at least two weeks. In some embodiments, the first period is at least three weeks. In some embodiments, the first period is at least four weeks. In some embodiments, the first period is at least five weeks. In some embodiments, the first period is at least six weeks. In some embodiments, the first period is at least one month. In some embodiments, the first period is at least three months. In some embodiments, the first period is at least six months.

In some embodiments, the lowest therapeutic dose (LTD) and the corresponding proposed dose and proposed dose range for the following compounds are as described in Table 2.

TABLE 2

| | Lowest | | Proposed | | Proposed | |
|---|---|---|---|---|---|---|
| | therapeutic | | Dose (mg) | | Dose Range | |
| Agent | dose (LTD) | | 25% TD | | 20%-30% TD | |
| Exenatide | 5 | mcg | 1.25 | mcg | 1-1.5 | mcg |
| Exenatide | 2 | mg | .5 | mg | .4-.6 | mg |
| (extended release) | | | | | | |
| Semaglutide (oral) | 7 | mg | 1.75 | mg | 1.4-2.1 | mg |
| Dulaglutide | .75 | mg | .1875 | mg | .150-.225 | mg |
| Liraglutide | 3 | mg | .75 | mg | .6-.9 | mg |
| Semaglutide | 2.4 | mg | .6 | mg | .48-.72 | mg |
| (injection) | | | | | | |

In some embodiments, the lowest therapeutic dose (LTD) and the corresponding proposed dose and proposed dose range for the following compounds are as described in Table 3.

TABLE 3

| | Lowest | | Proposed | | Proposed | |
|---|---|---|---|---|---|---|
| | therapeutic | | Dose (mg) | | Dose Range | |
| Agent | dose (LTD) | | 50% TD | | 45%-55% TD | |
| Exenatide | 5 | mcg | 2.5 | mcg | 2.25-2.75 | mcg |
| Exenatide | 2 | mg | 1 | mg | .9-1.1 | mg |
| (extended release) | | | | | | |
| Semaglutide (oral) | 7 | mg | 3.5 | mg | 3.15-3.85 | mg |
| Dulaglutide | .75 | mg | .375 | mg | .3375-.4125 | mg |
| Liraglutide | 3 | mg | 1.5 | mg | 1.35-1.65 | mg |
| Semaglutide | 2.4 | mg | 1.2 | mg | 1.08-1.32 | mg |
| (injection) | | | | | | |

In some embodiments, the lowest therapeutic dose (LTD) and the corresponding proposed dose and proposed dose range for the following compounds are as described in Table 4.

TABLE 4

| | Lowest | | Proposed | | Proposed | |
|---|---|---|---|---|---|---|
| | therapeutic | | Dose (mg) | | Dose Range | |
| Agent | dose (LTD) | | 75% TD | | 70%-80% TD | |
| Exenatide | 5 | mcg | 3.75 | mcg | 3.5-4 | mcg |
| Exenatide | 2 | mg | 1.5 | mg | 1.4-1.6 | mg |
| (extended release) | | | | | | |
| Semaglutide (oral) | 7 | mg | 5.25 | mg | 4.9-5.6 | mg |
| Dulaglutide | .75 | mg | .5625 | mg | .525-.6 | mg |
| Liraglutide | 3 | mg | 2.25 | mg | 2.1-2.4 | mg |
| Semaglutide | 2.4 | mg | 1.8 | mg | 1.68-1.92 | mg |
| (injection) | | | | | | |

In some embodiments, the methods of the present disclosure result in one or more of the following:
i. preventing, slowing progression of, delaying, or treating a metabolic disorder;
ii. preventing, slowing progression of, delaying, or treating diabetes;
iii. reducing body weight and/or body fat or preventing an increase in body weight and/or body fat or facilitating a reduction in body weight and/or body fat;
iv. preventing, slowing progression of, delaying or treating of a condition or disorder associated with obesity;
v. preventing, slowing progression of, delaying or treating of a condition or disorder associated with NAFLD;
vi. preventing, slowing progression of, delaying or treating of a condition or disorder associated with NASH;
vii. preventing, slowing progression of, or delaying of a condition or disorder associated with T2DM;
viii. preventing, slowing progression of, delaying or treating of a condition or disorder associated with hypertension;
ix. preventing, slowing progression of, delaying or treating of a condition or disorder associated with dyslipidemia;
x. preventing, slowing progression of, delaying or treating of a condition or disorder associated with osteoarthritis;
xi. preventing, slowing progression of, delaying or treating of a condition or disorder associated with kidney disease;
xii. preventing, slowing progression of, delaying or treating of a condition or disorder associated with sleep apnea;
xiii. preventing, slowing progression of, delaying or treating of a condition or disorder associated with PCOS;
xiv. preventing, slowing progression of, delaying, or treating obesity;
xv. preventing, slowing progression of, delaying, or treating NAFLD;
xvi. preventing, slowing progression of, delaying, or treating NASH;
xvii. preventing, slowing progression of, or delaying T2DM;
xviii. preventing, slowing progression of, delaying, or treating hypertension;
xix. preventing, slowing progression of, delaying, or treating dyslipidemia;
xx. preventing, slowing progression of, delaying, or treating osteoarthritis;
xxi. preventing, slowing progression of, delaying, or treating kidney disease;
xxii. preventing, slowing progression of, delaying, or treating sleep apnea;
xxiii. preventing, slowing progression of, delaying, or treating PCOS;
xxiv. preventing, slowing, delaying or treating diseases or conditions attributed to an abnormal accumulation of ectopic fat.

In some embodiments, the treatment results in a reduction in the weight or prevention of an increase of the weight in a patient in need thereof.

In some embodiments, the method results in efficacious treatment or prevention of obesity and/or obesity related diseases and disorders.

In some embodiments, treatment results in improved weight loss that is greater than the weight loss obtained with the full lowest therapeutic dose of any of the GLP-1 receptor agonists in the pharmaceutical composition.

In some embodiments, treatment results in greater long term tolerability and reduced risk of side effects when compared to treatment with the full lowest therapeutic dose of any of the GLP-1 receptor agonists in the pharmaceutical composition.

In some embodiments, the treatment is the initial or first-line treatment of obesity. In some embodiments, the subject has a body mass index (BMI) of less than 35. In some embodiments, the subject has a body mass index (BMI) of less than 30. In some embodiments, the subject has a body mass index (BMI) of more than 35. In some embodiments, the subject has a body mass index (BMI) of less than 40. In some embodiments, the subject has a body mass index (BMI) of between 30-35.

### Formulations

In some embodiments, the GLP-1 receptor agonist is provided in a formulation. In some embodiments, the formulation is provided as a pharmaceutical composition suitable for oral administration. In some embodiments, the formulation is provided as a pharmaceutical composition is in the form of pill, tablet, or capsule. In some embodiments, the formulation is provided as a pharmaceutical composition suitable for injection. In some embodiments, the formulation is provided as a pharmaceutical composition suitable for subcutaneous injection.

Other suitable formulations include, but are not limited to, those suitable for rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose.

Exemplary pharmaceutical compositions may be used in the form of a pharmaceutical preparation, for example, in solid, semisolid, or liquid form, which includes one or more GLP-1 receptor agonists, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills, and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. In some embodiments, capsules are prepared by encapsulating tablets in hard-gelatin capsules (e.g., over-encapsulation). Tablets, and other solid dosage forms, such as dragees, capsules, pills and granules, may optionally be scored or prepared with coatings and shells, such as enteric coatings and other coatings well known in the pharmaceutical-formulating art.

### Non-Limiting Embodiments

The following Embodiments are detailed by way of illustration only and are not to be construed as limiting in spirit or in scope of the present disclosure.
1. A method for treating or preventing obesity in a subject, comprising:
providing a glucagon-like peptide-1 (GLP-1) receptor agonist with a pharmaceutically or nutritionally acceptable carrier to the patient at a maintenance dose lower than a lowest therapeutic dose (LTD) to be administered during a first period; and
   wherein, during a second period that overlaps with the first period, the subject is on an insulin modulating nutritional therapy protocol.
2. The method of embodiment 1, wherein the insulin modulating nutritional therapy protocol comprises a hormonal nutritional therapy (HNT) protocol.
3. The method of any of embodiments 1-2, wherein the HNT protocol comprises reduced insulin fasting (RIF) protocol.
4. The method of any of embodiments 1-3, wherein the RIF protocol comprises an intermittent fasting protocol.
5. The method of any of embodiments 1-4, wherein the insulin modulating nutritional therapy protocol comprises a HNT protocol, and wherein the HNT protocol comprises a reduced insulin nutrition (RIN) protocol.
6. The method of any of embodiments 1-5, wherein the RIN protocol comprises a high protein and high natural fats diet.
7. The method of any of embodiments 1-6, wherein the maintenance dose is between 10%-95% of the LTD for the GLP-1 receptor agonist.
8. The method of any of embodiments 1-7, wherein the maintenance dose is between 65%-95% of the LTD for the GLP-1 receptor agonist.
9. The method of any of embodiments 1-8, wherein the maintenance dose is between 70%-90% of the LTD for the GLP-1 receptor agonist.
10. The method of any of embodiments 1-9, wherein the maintenance dose is between 70%-80% of the LTD for the GLP-1 receptor agonist.
11. The method of any of embodiments 1-10, wherein the maintenance dose is between 35%-65% of the LTD for the GLP-1 receptor agonist.
12. The method of any of embodiments 1-11, wherein the maintenance dose is between 40%-60% of the LTD for the GLP-1 receptor agonist.
13. The method of any of embodiments 1-12, wherein the maintenance dose is between 45%-55% of the LTD for the GLP-1 receptor agonist.
14. The method of any of embodiments 1-13, wherein the maintenance dose is between 10%-35% of the LTD for the GLP-1 receptor agonist.
15. The method of any of embodiments 1-14, wherein the maintenance dose is between 15%-30% of the LTD for the GLP-1 receptor agonist.
16. The method of any of embodiments 1-15, wherein the maintenance dose is between 15%-25% of the LTD for the GLP-1 receptor agonist.
17. The method of any of embodiments 1-16, wherein the GLP-1 receptor agonist comprises at least one of liraglutide, exenatide, lixisenatide, albiglutide, dulaglutide, semaglutide, taspoglutide, efpeglenatide, and tirzepatide.
18. The method of any of embodiments 1-17, wherein the GLP-1 receptor agonist comprises liraglutide.
19. The method of any of embodiments 1-18, wherein the GLP-1 receptor agonist comprises liraglutide and the maintenance dose is between 65-85% of the LTD for liraglutide.
20. The method of any of embodiments 1-19, wherein the GLP-1 receptor agonist comprises liraglutide and the maintenance dose is between 40-60% of the LTD for liraglutide.
21. The method of any of embodiments 1-20, wherein the GLP-1 receptor agonist comprises liraglutide and the maintenance dose is between 15-35% of the LTD for liraglutide.
22. The method of any of embodiments 1-21, wherein the GLP-1 receptor agonist comprises exenatide.
23. The method of any of embodiments 1-22, wherein the GLP-1 receptor agonist comprises exenatide and the maintenance dose is between 65-85% of the LTD for exenatide.
24. The method of any of embodiments 1-23, wherein the GLP-1 receptor agonist comprises exenatide and the maintenance dose is between 40-60% of the LTD for exenatide.
25. The method of any of embodiments 1-24, wherein the GLP-1 receptor agonist comprises exenatide and the maintenance dose is between 15-35% of the LTD for exenatide.
26. The method of any of embodiments 1-25, wherein the GLP-1 receptor agonist comprises lixisenatide.
27. The method of any of embodiments 1-26, wherein the GLP-1 receptor agonist comprises lixisenatide and the maintenance dose is between 65-85% of the LTD for lixisenatide.
28. The method of any of embodiments 1-27, wherein the GLP-1 receptor agonist comprises lixisenatide and the maintenance dose is between 40-60% of the LTD for lixisenatide.
29. The method of any of embodiments 1-28, wherein the GLP-1 receptor agonist comprises lixisenatide and the maintenance dose is between 15-35% of the LTD for lixisenatide.
30. The method of any of embodiments 1-29, wherein the GLP-1 receptor agonist comprises albiglutide.
31. The method of any of embodiments 1-30, wherein the GLP-1 receptor agonist comprises albiglutide and the maintenance dose is between 65-85% of the LTD for albiglutide.
32. The method of any of embodiments 1-31, wherein the GLP-1 receptor agonist comprises albiglutide and the maintenance dose is between 40-60% of the LTD for albiglutide.
33. The method of any of embodiments 1-32, wherein the GLP-1 receptor agonist comprises albiglutide and the maintenance dose is between 15-35% of the LTD for albiglutide.
34. The method of any of embodiments 1-33, wherein the GLP-1 receptor agonist comprises dulaglutide.
35. The method of any of embodiments 1-34, wherein the GLP-1 receptor agonist comprises dulaglutide and the maintenance dose is between 65-85% of the LTD for dulaglutide.
36. The method of any of embodiments 1-35, wherein the GLP-1 receptor agonist comprises dulaglutide and the maintenance dose is between 40-60% of the LTD for dulaglutide.
37. The method of any of embodiments 1-36, wherein the GLP-1 receptor agonist comprises dulaglutide and the maintenance dose is between 15-35% of the LTD for dulaglutide.
38. The method of any of embodiments 1-37, wherein the GLP-1 receptor agonist comprises semaglutide.
39. The method of any of embodiments 1-38, wherein the GLP-1 receptor agonist comprises semaglutide and the maintenance dose is between 65-85% of the LTD for semaglutide.
40. The method of any of embodiments 1-39, wherein the GLP-1 receptor agonist comprises semaglutide and the maintenance dose is between 40-60% of the LTD for semaglutide.
41. The method of any of embodiments 1-40, wherein the GLP-1 receptor agonist comprises semaglutide and the maintenance dose is between 15-35% of the LTD for semaglutide.
42. The method of any of embodiments 1-41, wherein the GLP-1 receptor agonist comprises taspoglutide.
43. The method of any of embodiments 1-42, wherein the GLP-1 receptor agonist comprises taspoglutide and the maintenance dose is between 65-85% of the LTD for taspoglutide.
44. The method of any of embodiments 1-43, wherein the GLP-1 receptor agonist comprises taspoglutide and the maintenance dose is between 40-60% of the LTD for taspoglutide.
45. The method of any of embodiments 1-44, wherein the GLP-1 receptor agonist comprises taspoglutide and the maintenance dose is between 15-35% of the LTD for taspoglutide.
46. The method of any of embodiments 1-45, wherein the GLP-1 receptor agonist comprises efpeglenatide.
47. The method of any of embodiments 1-46, wherein the GLP-1 receptor agonist comprises efpeglenatide and the maintenance dose is between 65-85% of the LTD for efpeglenatide.
48. The method of any of embodiments 1-47, wherein the GLP-1 receptor agonist comprises efpeglenatide and the maintenance dose is between 40-60% of the LTD for efpeglenatide.
49. The method of any of embodiments 1-48, wherein the GLP-1 receptor agonist comprises efpeglenatide and the maintenance dose is between 15-35% of the LTD for efpeglenatide.
50. The method of any of embodiments 1-49, wherein the GLP-1 receptor agonist comprises tirzepatide.
51. The method of any of embodiments 1-50, wherein the GLP-1 receptor agonist comprises tirzepatide and the maintenance dose is between 65-85% of the LTD for tirzepatide.
52. The method of any of embodiments 1-51, wherein the GLP-1 receptor agonist comprises tirzepatide and the maintenance dose is between 40-60% of the LTD for tirzepatide.
53. The method of any of embodiments 1-52, wherein the GLP-1 receptor agonist comprises tirzepatide and the maintenance dose is between 15-35% of the LTD for tirzepatide.
54. The method of any of embodiments 1-53, wherein providing the GLP-1 receptor agonist with the pharmaceutically or nutritionally acceptable carrier to the patient at the maintenance dose lower than the LTD to be administered during the first period is further provided to be administered using substantially the same dosing interval and treatment duration as recommended for the LTD.
55. The method of any of embodiments 1-54, wherein the obesity is associated with a disease or disorder, the method comprises preventing or treating the disease or disorder, and wherein the disease or disorder comprises one or more of nonalcoholic fatty liver disease (NAFLD), non-alcoholic steatohepatitis (NASH), type 2 diabetes mellitus (T2DM), hypertension, dyslipidemia, osteoarthritis, kidney disease, sleep apnea, and polycystic ovarian syndrome (PCOS).
56. The method of any of embodiments 1-55, further comprising providing at least one additional therapeutically active agent to be administered during the first period.
57. The method of any of embodiments 1-56, wherein the at least one additional therapeutically active agent comprises a biguanide, a dipeptidyl peptidase IV (DPP IV) inhibitor, and a sodium-glucose cotransporter-2 (SGLT-2) inhibitor.
58. The method of any of embodiments 1-57, wherein the maintenance dose is the lowest diabetes therapeutic dose (LDTD).
59. The method of any of embodiments 1-58, wherein providing the GLP-1 receptor agonist to the patient comprises providing the GLP-1 receptor agonist to the patient in accordance with a dose escalation schedule including the maintenance dose.
60. The method of any of embodiments 1-59, wherein the maintenance dose is higher than the LDTD but lower than the LOTD.
61. A method for treating or preventing obesity in a subject, comprising:
   providing a GLP-1 receptor agonist with a pharmaceutically or nutritionally acceptable carrier to the patient at a maintenance dose to be administered during a first period; and
   wherein, during a second period that overlaps with the first period, the subject is on an insulin modulating nutritional therapy protocol comprising at least one of a RIN and a RIF protocol.
62. The method of embodiment 61, wherein the RIF protocol comprises fasting at least 12 consecutive hours each day of the second period.
63. The method of any of embodiments 61-62, wherein the RIF protocol comprises skipping at least one meal.
64. The method of any of embodiments 61-63, wherein the at least 12 consecutive hours begins after breakfast and before at least one of lunch and dinner.
65. The method of any of embodiments 61-64, wherein the at least 12 consecutive hours begins after lunch and before dinner.
66. The method of any of embodiments 61-65, wherein the RIF protocol comprises fasting at least 16 consecutive hours each day of the second period.
67. The method of any of embodiments 61-66, wherein the RIF protocol comprises fasting at least 20 consecutive hours each day of the second period.
68. The method of any of embodiments 61-67, wherein the RIF protocol comprises fasting at least 23 consecutive hours each day of the second period.
69. The method of any of embodiments 61-68, wherein the RIF protocol comprises an intermittent fasting protocol.
70. The method of any of embodiments 61-69, wherein the RIN protocol requires less than 25% of daily calories consumed by the subject to be from carbohydrates.
71. The method of any of embodiments 61-70, wherein the RIN protocol requires less than 20% of daily calories consumed by the subject to be from carbohydrates.
72. The method of any of embodiments 61-71, wherein the RIN protocol requires less than 15% of daily calories consumed by the subject to be from carbohydrates.
73. The method of any of embodiments 61-72, wherein the RIN protocol requires less than 10% of daily calories consumed by the subject to be from carbohydrates.
74. The method of any of embodiments 61-73, wherein the maintenance dose is a dose lower than a LTD.
75. The method of any of embodiments 61-74, wherein the maintenance dose is between 10%-95% of the LTD for the GLP-1 receptor agonist.
76. The method of any of embodiments 61-75, wherein the maintenance dose is between 65%-95% of the LTD for the GLP-1 receptor agonist.
77. The method of any of embodiments 61-76, wherein the maintenance dose is between 70%-90% of the LTD for the GLP-1 receptor agonist.
78. The method of any of embodiments 61-77, wherein the maintenance dose is between 70%-80% of the LTD for the GLP-1 receptor agonist.
79. The method of any of embodiments 61-78, wherein the maintenance dose is between 35%-65% of the LTD for the GLP-1 receptor agonist.
80. The method of any of embodiments 61-79, wherein the maintenance dose is between 40%-60% of the LTD for the GLP-1 receptor agonist.
81. The method of any of embodiments 61-80, wherein the maintenance dose is between 45%-55% of the LTD for the GLP-1 receptor agonist.
82. The method of any of embodiments 61-81, wherein the maintenance dose is between 10%-35% of the LTD for the GLP-1 receptor agonist.
83. The method of any of embodiments 61-82, wherein the maintenance dose is between 15%-30% of the LTD for the GLP-1 receptor agonist.
84. The method of any of embodiments 61-83, wherein the maintenance dose is between 15%-25% of the LTD for the GLP-1 receptor agonist.
85. The method of any of embodiments 61-84, wherein the GLP-1 receptor agonist comprises at least one of liraglutide, exenatide, lixisenatide, albiglutide, dulaglutide, semaglutide, taspoglutide, efpeglenatide, and tirzepatide.
86. The method of any of embodiments 61-85, wherein the obesity is associated with a disease or disorder, the method comprises preventing or treating the disease or disorder, and wherein the disease or disorder comprises one or more of nonalcoholic fatty liver disease (NAFLD), non-alcoholic steatohepatitis (NASH), type 2 diabetes mellitus (T2DM), hypertension, dyslipidemia, osteoarthritis, kidney disease, sleep apnea, and polycystic ovarian syndrome (PCOS).
87. The method of any of embodiments 61-86, further comprising providing at least one additional therapeutically active agent to be administered during the first period.
88. The method of any of embodiments 61-87, wherein the at least one additional therapeutically active agent comprises a biguanide, a dipeptidyl peptidase IV (DPP IV) inhibitor, and a sodium-glucose cotransporter-2 (SGLT-2) inhibitor.
89. The method of any of embodiments 61-88, wherein the maintenance dose is the lowest diabetes therapeutic dose (LDTD).
90. The method of any of embodiments 61-89, wherein providing the GLP-1 receptor agonist to the patient comprises providing the GLP-1 receptor agonist to the patient in accordance with a dose escalation schedule including the maintenance dose.
91. The method of any of embodiments 61-90, wherein the maintenance dose is higher than the LDTD but lower than the LOTD.
92. A method for treating or preventing obesity in a subject, comprising:
   providing a GLP-1 receptor agonist with a pharmaceutically or nutritionally acceptable carrier to the patient at a maintenance dose to be administered during a first period; and
   wherein, during a second period that overlaps with the first period, the subject is on an insulin modulating nutritional therapy protocol comprising at least one of a RIN and a RIF protocol.
93. The method of embodiment 92, wherein the second period is at least two weeks.
94. The method of any of embodiments 92-93, wherein the second period is at least 3 weeks.
95. The method of any of embodiments 92-94, wherein the second period is at least 4 weeks.
96. The method of any of embodiments 92-95, wherein the second period is at least 5 weeks.
97. The method of any of embodiments 92-96, wherein the second period is at least 6 weeks.
98. The method of any of embodiments 1-97, wherein the first period is entirely within the second period.
99. The method of any of embodiments 1-98, wherein the first period and the second period overlap at least 1 week.
100. The method of any of embodiments 1-99, wherein the first period and the second period overlap at least 1 week.
101. The method of any of embodiments 1-100, wherein the first period and the second period overlap at least 1 week.
102. The method of any of embodiments 1-101, wherein the first period and the second period overlap at least 2 weeks.
103. The method of any of embodiments 1-102, wherein the first period and the second period overlap at least 3 weeks.
104. The method of any of embodiments 1-103, wherein the first period and the second period overlap at least 1 month.
105. The method of any of embodiments 1-104, wherein the first period and the second period overlap at least 3 months.
106. The method of any of embodiments 1-105, wherein the first period is at least 1 week.
107. The method of any of embodiments 1-106, wherein the first period is at least 2 weeks.
108. The method of any of embodiments 1-107, wherein the first period is at least 3 weeks.
109. The method of any of embodiments 1-108, wherein the first period is at least 4 weeks.
110. The method of any of embodiments 1-109, wherein the first period is at least 1 month.
111. The method of any of embodiments 1-110, wherein the first period is at least 3 months.
112. The method of any of embodiments 1-111, wherein the second period is at least 1 week.

### Non-Limiting Examples

The example below is provided to give those of ordinary skill in the art with a complete disclosure and description of how to make and use the claimed embodiments, and is not intended to limit the scope of what is disclosed herein. Modifications that are obvious to persons of skill in the art are intended to be within the scope of the following claims.

### Example 1—Clinical Study of Combining Semaglutide (Injection) and Fasting

Patient 1 was subject to a combination HMT/HNT treatment protocol as described below. As shown in Table 5, patient 1 lost 30 pounds in 30 days, resulting in an approximately 9.6% bodyweight reduction, The results were achieved without exercise, without caloric restrictions or calorie counting, and with no discernable or unusual hunger.

TABLE 5

| Patient | 1 | |
|---|---|---|
| Starting Weight | 282 | lbs |
| Ending Weight | 252 | lbs |
| Treatment Duration | 30 | days |
| Bodyweight reduction | −30 | lbs |

| | |
|---|---|
| % Bodyweight Reduction | −9.6% |

Hormonal Nutritional Therapy

| Fasting Protocol Achieved | One-Meal-A-Day | |
|---|---|---|
| Time to achieve fasting protocol | 1 | week |

Hormonal Medical Therapy

| | |
|---|---|
| Treatment utilized | Semaglutide |
| Dose | Week 1: .25 mg |
| | Week 2: .50 mg |
| | Week 3: .50 mg |
| | Week 4: 1 mg |
| Other Notes | Withing's Smart scale and App |
| | No calorie counting or |
| | targeted caloric restriction |
| | No exercise |
| | No discernable/unusual hunger |

### Example 2—Clinical Study of Combining Semaglutide (injection) and Fasting

Patients are subject to a combination HMT/HNT treatment protocol as described in Table 6 below. Desirable therapeutics effects (e.g. weigh loss) are achieved without exercise, without caloric restrictions or calorie counting, and with no discernable or unusual hunger.

TABLE 6
Hormonal Nutritional Therapy

| | |
|---|---|
| Fasting Protocol Achieved | 15-hour fasting (no calorie counting |
| | or targeted caloric restriction) |
| Time to achieve fasting protocol | 1 week |

Hormonal Medical Therapy

| | |
|---|---|
| Treatment utilized | Semaglutide |
| Dose | Week 1: .25 mg |
| | Week 2: .50 mg |
| | Week 3: .50 mg |
| | Week 4: 1 mg |

### Example 3—Clinical Study of Combining Semaglutide (oral) and Fasting

Patients are subject to a combination HMT/HNT treatment protocol as described in Table 7 below. Desirable therapeutics effects (e.g. weigh loss) are achieved without exercise, without caloric restrictions or calorie counting, and with no discernable or unusual hunger.

TABLE 7
Hormonal Nutritional Therapy

| | |
|---|---|
| Fasting Protocol Achieved | 18-hour fasting (no calorie counting |
| | or targeted caloric restriction) |
| Time to achieve fasting protocol | 1 week |

Hormonal Medical Therapy

| | |
|---|---|
| Treatment utilized | Semaglutide |
| Dose | Week 1: .25 mg |
| | Week 2: .50 mg |
| | Week 3: .50 mg |
| | Week 4: 1 mg |

### Example 4—Clinical Study of Combining Semaglutide (Oral) and Fasting

Patients are subject to a combination HMT/HNT treatment protocol as described in Table 8 below. Desirable therapeutics effects (e.g. weigh loss) are achieved without exercise, without caloric restrictions or calorie counting, and with no discernable or unusual hunger.

TABLE 8
Hormonal Nutritional Therapy

| | |
|---|---|
| Fasting Protocol Achieved | One-Meal-A-Day (no calorie counting |
| | or targeted caloric restriction) |
| Time to achieve fasting protocol | 1 week |

Hormonal Medical Therapy

| | |
|---|---|
| Treatment utilized | Semaglutide (oral) |
| Dose | Week 1: 1 mg |
| | Week 2: 2 mg |
| | Week 3: 3.5 mg |
| | Week 4: 5 mg |

### Example 5—Clinical Study of Combining Semaglutide (Oral) and Fasting

Patients are subject to a combination HMT/HNT treatment protocol as described in Table 9 below. Desirable therapeutics effects (e.g. weigh loss) are achieved without exercise, without caloric restrictions or calorie counting, and with no discernable or unusual hunger.

TABLE 9
Hormonal Nutritional Therapy

| | |
|---|---|
| Fasting Protocol Achieved | 15-hour fasting (no calorie counting |
| | or targeted caloric restriction) |
| Time to achieve fasting protocol | 1 week |

Hormonal Medical Therapy

| | |
|---|---|
| Treatment utilized | Semaglutide (oral) |
| Dose | Week 1: 1 mg |
| | Week 2: 2 mg |
| | Week 3: 3.5 mg |
| | Week 4: 5 mg |

### Example 6—Clinical Study of Combining Semaglutide (Oral) and Fasting

Patients are subject to a combination HMT/HNT treatment protocol as described in Table 10 below. Desirable therapeutics effects (e.g. weigh loss) are achieved without exercise, without caloric restrictions or calorie counting, and with no discernable or unusual hunger.

TABLE 10
Hormonal Nutritional Therapy

| | |
|---|---|
| Fasting Protocol Achieved | 18-hour fasting (no calorie counting |
| | or targeted caloric restriction) |
| Time to achieve fasting protocol | 1 week |

Hormonal Medical Therapy

| | |
|---|---|
| Treatment utilized | Semaglutide (oral) |
| Dose | Week 1: 1 mg |
| | Week 2: 2 mg |
| | Week 3: 3.5 mg |
| | Week 4: 5 mg |

### Example 7—Clinical Study of Combining Exenatide (Regular) and Fasting

Patients are subject to a combination HMT/HNT treatment protocol as described in Table 11 below. Desirable therapeutics effects (e.g. weigh loss) are achieved without exercise, without caloric restrictions or calorie counting, and with no discernable or unusual hunger.

TABLE 11
Hormonal Nutritional Therapy

| | |
|---|---|
| Fasting Protocol Achieved | One-Meal-A-Day (no calorie counting |
| | or targeted caloric restriction) |
| Time to achieve fasting protocol | 1 week |

Hormonal Medical Therapy

| | |
|---|---|
| Treatment utilized | Exenatide (regular) |
| Dose | Week 1: 1.25 mg |
| | Week 2: 2.5 mg |
| | Week 3: 3.75mg |
| | Week 4: 3.75 mg |

### Example 8—Clinical Study of Combining Exenatide (Regular) and Fasting

Patients are subject to a combination HMT/HNT treatment protocol as described in Table 12 below. Desirable therapeutics effects (e.g. weigh loss) are achieved without exercise, without caloric restrictions or calorie counting, and with no discernable or unusual hunger.

TABLE 12
Hormonal Nutritional Therapy

| | |
|---|---|
| Fasting Protocol Achieved | 15-hour fasting (no calorie counting |
| | or targeted caloric restriction) |
| Time to achieve fasting protocol | 1 week |

Hormonal Medical Therapy

| | |
|---|---|
| Treatment utilized | Exenatide (regular) |
| Dose | Week 1: 1.25 mg |
| | Week 2: 2.5 mg |
| | Week 3: 3.75mg |
| | Week 4: 3.75 mg |

### Example 9—Clinical Study of Combining Exenatide (Regular) and Fasting

Patients are subject to a combination HMT/HNT treatment protocol as described in Table 13 below. Desirable therapeutics effects (e.g. weigh loss) are achieved without exercise, without caloric restrictions or calorie counting, and with no discernable or unusual hunger.

TABLE 13
Hormonal Nutritional Therapy

| | |
|---|---|
| Fasting Protocol Achieved | 18-hour fasting (no calorie counting |
| | or targeted caloric restriction) |
| Time to achieve fasting protocol | 1 week |

Hormonal Medical Therapy

| | |
|---|---|
| Treatment utilized | Exenatide (regular) |
| Dose | Week 1: 1.25 mg |
| | Week 2: 2.5 mg |
| | Week 3: 2.5 mg |
| | Week 4: 3.75 mg |

### Example 10—Clinical Study of Combining Exenatide (Extended Release) and Fasting

Patients are subject to a combination HMT/HNT treatment protocol as described in Table 14 below. Desirable therapeutics effects (e.g. weigh loss) are achieved without exercise, without caloric restrictions or calorie counting, and with no discernable or unusual hunger.

TABLE 14
Hormonal Nutritional Therapy

| | |
|---|---|
| Fasting Protocol Achieved | One-Meal-A-Day (no calorie counting |
| | or targeted caloric restriction) |
| Time to achieve fasting protocol | 1 week |

Hormonal Medical Therapy

| | |
|---|---|
| Treatment utilized | Exenatide (extended release) |
| Dose | Week 1: 0.5 mg |
| | Week 2: 1 mg |
| | Week 3: 1 mg |
| | Week 4: 1.5 mg |

### Example 11—Clinical Study of Combining Exenatide (Extended Release) and Fasting

Patients are subject to a combination HMT/HNT treatment protocol as described in Table 15 below. Desirable therapeutics effects (e.g. weigh loss) are achieved without exercise, without caloric restrictions or calorie counting, and with no discernable or unusual hunger.

TABLE 15
Hormonal Nutritional Therapy

| | |
|---|---|
| Fasting Protocol Achieved | 15-hour fasting (no calorie counting |
| | or targeted caloric restriction) |
| Time to achieve fasting protocol | 1 week |

Hormonal Medical Therapy

| | |
|---|---|
| Treatment utilized | Exenatide (extended release) |
| Dose | Week 1: 0.5 mg |
| | Week 2: 1 mg |
| | Week 3: 1.5 mg |
| | Week 4: 1.5 mg |

### Example 12—Clinical Study of Combining Exenatide (Extended Release) and Fasting

Patients are subject to a combination HMT/HNT treatment protocol as described in Table 16 below. Desirable therapeutics effects (e.g. weigh loss) are achieved without exercise, without caloric restrictions or calorie counting, and with no discernable or unusual hunger.

TABLE 16
Hormonal Nutritional Therapy

| | |
|---|---|
| Fasting Protocol Achieved | 18-hour fasting (no calorie counting |
| | or targeted caloric restriction) |
| Time to achieve fasting protocol | 1 week |

Hormonal Medical Therapy

| | |
|---|---|
| Treatment utilized | Exenatide (regular) |
| Dose | Week 1: 0.5 mg |
| | Week 2: 1 mg |
| | Week 3: 1 mg |
| | Week 4: 1.5 mg |

### Example 13—Clinical Study of Combining Dulaglutide and Fasting

Patients are subject to a combination HMT/HNT treatment protocol as described in Table 17 below. Desirable therapeutics effects (e.g. weigh loss) are achieved without exercise, without caloric restrictions or calorie counting, and with no discernable or unusual hunger.

TABLE 17
Hormonal Nutritional Therapy

| | |
|---|---|
| Fasting Protocol Achieved | One-Meal-A-Day (no calorie counting |
| | or targeted caloric restriction) |
| Time to achieve fasting protocol | 1 week |

Hormonal Medical Therapy

| | |
|---|---|
| Treatment utilized | Dulaglutide |
| Dose | Week 1: 0.2 mg |
| | Week 2: 0.375 mg |
| | Week 3: 0.375 mg |
| | Week 4: 0.6 mg |

### Example 14—Clinical Study of Combining Dulaglutide and Fasting

Patients are subject to a combination HMT/HNT treatment protocol as described in Table 18 below. Desirable therapeutics effects (e.g. weigh loss) are achieved without exercise, without caloric restrictions or calorie counting, and with no discernable or unusual hunger.

TABLE 18
Hormonal Nutritional Therapy

| | |
|---|---|
| Fasting Protocol Achieved | 15-hour fasting (no calorie counting |
| | or targeted caloric restriction) |
| Time to achieve fasting protocol | 1 week |

Hormonal Medical Therapy

| | |
|---|---|
| Treatment utilized | Dulaglutide |
| Dose | Week 1: 0.2 mg |
| | Week 2: 0.375 mg |
| | Week 3: 0.375 mg |
| | Week 4: 0.6 mg |

### Example 15—Clinical Study of Combining Dulaglutide and Fasting

Patients are subject to a combination HMT/HNT treatment protocol as described in Table 19 below. Desirable therapeutics effects (e.g. weigh loss) are achieved without exercise, without caloric restrictions or calorie counting, and with no discernable or unusual hunger.

TABLE 19
Hormonal Nutritional Therapy

| | |
|---|---|
| Fasting Protocol Achieved | 18-hour fasting (no calorie counting |
| | or targeted caloric restriction) |
| Time to achieve fasting protocol | 1 week |

Hormonal Medical Therapy

| | |
|---|---|
| Treatment utilized | Dulaglutide |
| Dose | Week 1: 0.2 mg |
| | Week 2: 0.375 mg |
| | Week 3: 0.375 mg |
| | Week 4: 0.6 mg |

### Example 16—Clinical Study of Combining Liraglutide and Fasting

Patients are subject to a combination HMT/HNT treatment protocol as described in Table 20 below. Desirable therapeutics effects (e.g. weigh loss) are achieved without exercise, without caloric restrictions or calorie counting, and with no discernable or unusual hunger.

TABLE 20
Hormonal Nutritional Therapy

| | |
|---|---|
| Fasting Protocol Achieved | One-Meal-A-Day (no calorie counting |
| | or targeted caloric restriction) |
| Time to achieve fasting protocol | 1 week |

Hormonal Medical Therapy

| | |
|---|---|
| Treatment utilized | Liraglutide |
| Dose | Week 1: 0.75 mg |
| | Week 2: 1.5 mg |
| | Week 3: 1.5 mg |
| | Week 4: 2.25 mg |

### Example 17—Clinical Study of Combining Liraglutide and Fasting

Patients are subject to a combination HMT/HNT treatment protocol as described in Table 21 below. Desirable therapeutics effects (e.g. weigh loss) are achieved without exercise, without caloric restrictions or calorie counting, and with no discernable or unusual hunger.

TABLE 21
Hormonal Nutritional Therapy

| | |
|---|---|
| Fasting Protocol Achieved | 15-hour fasting (no calorie counting |
| | or targeted caloric restriction) |
| Time to achieve fasting protocol | 1 week |

Hormonal Medical Therapy

| | |
|---|---|
| Treatment utilized | Liraglutide |
| Dose | Week 1: 0.75 mg |
| | Week 2: 1.5 mg |
| | Week 3: 2.25 mg |
| | Week 4: 2.25 mg |

### Example 18—Clinical Study of Combining Liraglutide and Fasting

Patients are subject to a combination HMT/HNT treatment protocol as described in Table 22 below. Desirable therapeutics effects (e.g. weigh loss) are achieved without exercise, without caloric restrictions or calorie counting, and with no discernable or unusual hunger.

TABLE 22
Hormonal Nutritional Therapy

| | |
|---|---|
| Fasting Protocol Achieved | 18-hour fasting (no calorie counting |
| | or targeted caloric restriction) |
| Time to achieve fasting protocol | 1 week |

Hormonal Medical Therapy

| | |
|---|---|
| Treatment utilized | Liraglutide |
| Dose | Week 1: 0.75 mg |
| | Week 2: 1.5 mg |
| | Week 3: 1.5 mg |
| | Week 4: 2.25 mg |

While some preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for treating obesity in a patient, comprising:
administering a pharmaceutical composition comprising a glucagon-like peptide-1 (GLP-1) receptor agonist and a pharmaceutically acceptable carrier to the patient at a maintenance dose being 10% to 55% of a lowest therapeutic dose (LTD) to be administered during a first period; and
during a second period that overlaps with the first period, subjecting the patient to an intermittent fasting protocol, wherein the intermittent fasting protocol comprises fasting at least 12 consecutive hours each day of the second period.

2. The method of claim 1, wherein the intermittent fasting protocol comprises fasting at least 15 consecutive hours each day of the second period.

3. The method of claim 1, wherein the intermittent fasting protocol comprises fasting at least 16 consecutive hours each day of the second period.

4. The method of claim 1, wherein the intermittent fasting protocol comprises fasting at least 17 consecutive hours each day of the second period.

5. The method of claim 1, wherein the intermittent fasting protocol comprises fasting at least 18 consecutive hours each day of the second period.

6. The method of claim 1, wherein the intermittent fasting protocol comprises fasting at least 20 consecutive hours each day of the second period.

7. The method of claim 1, wherein the intermittent fasting protocol comprises fasting at least 23 consecutive hours each day of the second period.

8. The method of claim 1, wherein the maintenance dose is between 35%-55% of the LTD for the GLP-1 receptor agonist.

9. The method of claim 1, wherein the maintenance dose is between 45%-55% of the LTD for the GLP-1 receptor agonist.

10. The method of claim 1, wherein the maintenance dose is between 10%-35% of the LTD for the GLP-1 receptor agonist.

11. The method of claim 1, wherein the maintenance dose is between 15%-25% of the LTD for the GLP-1 receptor agonist.

12. The method of claim 1, wherein the GLP-1 receptor agonist is selected from the group consisting of liraglutide, exenatide, lixisenatide, albiglutide, dulaglutide, semaglutide, taspoglutide, efpeglenatide, tirzepatide, and combinations thereof.

13. The method of claim 1, wherein the GLP-1 receptor agonist comprises semaglutide.

14. The method of claim 1, wherein the GLP-1 receptor agonist comprises semaglutide and the maintenance dose is between 40%-55% of the LTD for semaglutide.

15. The method of claim 1, wherein the GLP-1 receptor agonist comprises semaglutide and the maintenance dose is between 15%-35% of the LTD for semaglutide.

16. The method of claim 1, wherein providing the GLP-1 receptor agonist to the patient comprises providing the GLP-1 receptor agonist to the patient in accordance with a dose escalation schedule including the maintenance dose.

17. The method of claim 1, wherein the second period is at least two weeks.

18. The method of claim 1, wherein the second period is at least 3 weeks.

19. The method of claim 1, wherein the second period is at least 4 weeks.

20. The method of claim 1, wherein the first period is entirely within the second period.
